(19) United States
(12) Patent Application Publication
(10) Pub. No.: US 2010/0003342 A1
(43) Pub. Date: Jan. 7, 2010

(54) Aqueous solution and method of prolonging life of residual chlorine in aqueous solution

(75) Inventor: Hidetaka Ito, Chiba (JP)

Correspondence Address: JORDAN AND HAMBURG LLP, 122 EAST 42ND STREET, SUITE 4000, NEW YORK, NY, 10168, US

(73) Assignee: Ideo Co., Ltd., Matsudo-shi (JP)

(21) Appl. No.: 12/312,939

(22) PCT Filed: May 29, 2007
(86) PCT No.: PCT/JP2007/060868

§ 371 Date: Jun. 1, 2009

(30) Foreign Application Priority Data
Dec. 11, 2006 (JP) 2006-333815

(51) Int. Cl. A01N 59/00; C11D 7/18; A01P 1/00

(52) U.S. Cl. 424/616; 424/661; 424/613; 510/379

(57) ABSTRACT

An aqueous solution which retains a high residual chlorine concentration over a long life and has excellent disinfectant (bactericidal) ability. The aqueous solution contains at least one member selected from the group consisting of halogen acids and salts thereof and further contains active oxygen, wherein the halogen acids are at least one member selected from the group consisting of hypochlorous acid, chlorous acid, chloric acid, and perchloric acid, the total amount of the at least one member selected from the group consisting of the halogen acids and salts thereof and contained in the aqueous solution is 10-50,000 ppm, and the total amount of the active oxygen contained in the aqueous solution is 0.1-1,000 ppm.

### APPLICABLE FIELD IN THE INDUSTRY

The present invention relates to an aqueous solution. The present invention more particularly relates to a technology of prolonging life duration of residual chlorine, being an index for indicating an ability (a sterilizing ability, a disinfectant ability, or an oxidizing ability) that is required for a disinfectant solution or a cleaning solution.

### BACKGROUND ART

A hypohalite (in particular, a hypochlorite (ClO⁻)) aqueous solution is used for disinfectant (sterilization) etc. The oxidizing ability of this hypochlorite (the disinfectant ability: the sterilizing ability) is evaluated by means of a concentration of residual hypochlorous acid. The concentration of the hypochlorous acid is evaluated by means of a residual chlorine concentration. And, the residual chlorine concentration is measured with an iodine method or a DPD (diethyl-p-phenylenediamine) method.

By the way, life duration of the residual chlorine owing to the hypochlorous acid is short. Life duration of this residual chlorine is dependent upon pH. And, as acidity becomes higher, chlorine gas is generated all the more (see Equation [1] and Equation [2]). This chlorine gas is volatized. Thus, as acidity becomes higher, life duration of the hypochlorous acid becomes shorter. Further, a generated chlorine molecule causes oxygen to occur. The hypochlorous acid is decomposed due to this oxygen. Thus, increasingly, life duration of the hypochlorous acid becomes shorter.

2HClO+2H⁺+2e⁻ [symbol] Cl₂+2H₂O Equation [1]

Cl₂+2H₂O [symbol] 2H⁺+2Cl⁻+O₂ Equation [2]

Additionally, when the aqueous solution is alkalinized, seemingly, life duration of the residual chlorine is prolonged. There can be listed the following two reasons as a main factor. One reason is that occurrence of chlorine gas is suppressed (see Equation [1]). The other reason is that the hypochlorous acid is changed to chloric acid that is stable (see Equation [3]).

3HClO [symbol] HClO₃+2HCl Equation [3]

This reaction (Equation [3]) progresses as alkalinity is raised, and resultantly, the chloric acid concentration becomes higher. That is the reason why, in many cases, the pH of commodities containing the hypochlorous acid is made alkalinic. Additionally, the chloric acid itself does not contribute to a concentration of the residual chlorine, so more alkalinic the pH is made, smaller the residual chlorine concentration becomes.

### DISCLOSURE OF THE INVENTION

### Problems to be Solved by the Invention

By the way, as described above, a disinfectant (sterilizing) operation of the hypohalite, in particular, the hypochlorite (ClO⁻) is greatly affected by a concentration of ClO⁻. That is, the disinfectant (sterilizing) ability is greatly affected by life duration of ClO⁻.

Incidentally, the fact that life duration of Co is short means that it is impossible to produce the cleaning solution in advance and to leave it as it is. In other words, it means that it is difficult to utilize the pre-produced cleaning solution (ClO⁻-containing aqueous solution).

Accordingly, the fact that life duration of ClO⁻ is long means that it becomes possible to produce the cleaning solution (ClO⁻-containing aqueous solution) in advance and to leave it as it is. This enables a cost for producing the cleaning solution to be reduced, and the cleaning solution to be effectively used.

Thus, the present invention has been accomplished so as to solve the above-mentioned problems, and an object thereof is to provide a technology of prolonging life duration of an oxidizing ability (disinfectant ability: sterilizing ability) of ClO⁻ etc.

### Means for Solving the Problem

By the way, HClO₃, originally, is a compound of which the oxidizing ability is higher than that of HClO. However, HClO₃ is small in a reaction speed at a room temperature, and seemingly, HClO₃ does not react. Additionally, nowadays, HClO₃ has not been isolated yet. It has been only isolated as a form of a chlorate. And, HClO₃ is stabilized at a room temperature. However, the chlorate is decomposed due to heat (see equation [4] and Equation [5]).

4MClO₃→3MClO₄+MCl Equation [4]

MClO₄→MCl+2O₂ Equation [5]

Incidentally, it is impossible to measure a concentration of the chlorate with a residual chlorine concentration measuring method. This is unchanged even though pH is changed from an acidity level to an alkalinity level.

HClO₄ is higher in a potential oxidizing ability than HClO₃. However, HClO₄, similarly to HClO₃, is small in a reaction speed at a room temperature, and seemingly, an oxidation reaction does not occur. And, a measurement with a KI method demonstrates that the residual chlorine concentration of the perchloric acid aqueous solution is zero.

A measurement with the KI method demonstrates that the residual chlorine concentration of NaClO₂ as well is several ppm in a neutral pH environment. NaClO₂ is not so high in the reactivity. However, when an aqueous solution of a chlorite is acidified, ClO₂ is generated (see equation [6]). And, the residual chlorine concentration is raised.

5NaClO₂+4HCl→4ClO₂+5NaCl+2H₂O Equation [6]

By the way, HClO and ClO₂ are known as a chemical species that contributes to the residual chlorine concentration. However, life duration of HClO and ClO₂, which are high in safetiness, is short. Thus, it is difficult to bottle and sale theses aqueous solutions. Thereupon, so as to extend life duration of the aqueous solution, it is desirable to cause the chemical species (the chemical species that makes it possible to supplement HClO or ClO₂ when HClO and ClO₂ are exhausted) to coexist therewith.

The case of HClO₃ and HClO will be explained as a typified example of this idea. As apparent from Equation 3, HClO and HClO₃ have a reversible relation with each other. This is a reaction that is called a reaction of ununiformization of HClO. That is, increasing a HClO₃ concentration leads to an increase in a HClO concentration. However, when HClO has not been dissolved, generation of HClO is not observed even though the aqueous solution having a chlorate dissolved therein is acidified.

Incidentally, when OH. (radical) coexists with the HClO₃ aqueous solution, ClO₂ of which the residual chlorine concentration can be measured is generated (see Equation [7]). Further, also in the case of causing the active oxygen such as H₂O₂ and superoxide anion to coexist therewith, ClO₂ of which the residual chlorine concentration can be measured is be generated (see Equation [8]).

ClO₃⁻+OH.+3H⁺→ClO₂+2H₂O Equation [7]

2ClO₃⁻+O₂⁻+8H⁺+5e⁻→2ClO₂+4H₂O Equation [8]

That is, adding the chlorate to the hypochlorite aqueous solution, and yet causing the active oxygen to coexist therewith lead to an increase in the actual concentration of the residual chlorine owing to ClO₂, or HClO etc.

By the way, when NaClO₃ and KClO₃ are caused to be dissolved in water, a chlorate aqueous solution is obtained. HClO₃ aqueous solution is also obtained with electrolysis. For example, as shown in FIG. 1, electrolytic water obtained by an electrolysis device having a two-chamber electrolysis cell (an electrolysis cell employing a fluorine-based cation exchange membrane as a membrane between an anode polarity and a cathode polarity) is high in a concentration of ozone (active oxygen) (JP-P1996-134677A, and JP-P2000-234191A). In FIG. 1, 1 is an anode chamber, and 2 is an inlet of the anode chamber. 3 is an exit of the anode chamber. 4 is an anode electrode. 5 is a membrane. 6 is a cathode chamber. 7 is an inlet of the cathode chamber. 8 is an exit of the cathode chamber. 9 is a cathode polarity. And, O₃ that has occurred, and Cl⁻ react with each other, thereby generating ClO₃⁻ (see Equation [9]).

Cl⁻+O₃→ClO₃⁻ Equation [9]

It has been understood that combining the active oxygen with this generated chlorous acid aqueous solution enables an oxidizing aqueous solution having a long life duration of the residual chlorine concentration to be obtained. Additionally, the active oxygen is generated with electrolysis. ClO⁻ is generated by subjecting Cl⁻ to an anodic electrolysis oxidation. For example, when a salt of NaCl etc. is added to the cathode chamber 6 of the two-chamber electrolysis cell of FIG. 1, one part of Cl⁻ becomes Cl₂, and one part thereof, which reacts with the generated O₃, becomes ClO₃⁻.

And, when ClO₃⁻ is oxidized, ClO₄⁻ is generated (see Equation [10] and Equation [11]).

ClO₃⁻+H₂O−2e⁻→ClO₄⁻+2H⁺ Equation [10]

ClO₃⁻+O.→ClO₄⁻ Equation [11]

Additionally, in the above-mentioned electrolysis device, a fluorine-based cation exchange membrane was used as the membrane (porous membrane) that closely stuck to the anode electrode 4. Similarly to the case of employing the foregoing two-chamber electrolysis cell, when the halogen salt aqueous solution is supplied to an electrolyte supplementing chamber, thereby to anode-oxidize a halogen salt, a high-order halogen acid is generated. The residual chlorine concentration can be raided because the active oxygen as well is simultaneously generated.

Further, when the electrolysis is carried out by supplying saline water to an intermediate chamber 11 of a three-chamber electrolysis cell (the three-chamber electrolysis cell includes the intermediate chamber 11 between an anode chamber 1 and a cathode chamber 9. See FIG. 2), and pure water to the anode chamber 1 and the cathode chamber 9, respectively, ozone etc. is generated in the anode chamber 1. Yet, dissolved oxygen is reduced in the cathode chamber 9, and active oxygen (O₂⁻) is generated. This active oxygen allows the residual chlorine concentration of the HClO₃ aqueous solution to be raised. In FIG. 2, 2 is an inlet of an anode chamber. 3 is an exit of the anode chamber. 4 is an anode electrode. Each of 5 and 6 is a membrane. 7 is a cathode electrode. 8 is an exit of the cathode chamber. 10 is an inlet of the cathode chamber. 12 is an inlet of the intermediate chamber. 13 is an exit of the intermediate chamber.

Thus, so as to prolong life duration of the residual chlorine, it is important to generate HClO₂, HClO₃ and/or HClO₄ each of which is a higher-order oxide as compared with HClO. So as to generate a higher-order oxide, it is important to raise an occurrence efficiency of an oxygen-based oxide such as O₃ etc. and enhancing an efficiency of a direct reaction with Cl⁻ etc. The electrode surface and the vicinity of the electrode, in which gas of oxygen etc. occurs with a progress of the anodic electrolysis oxidation of water, is under a vapor phase environment. Thus, it is preferable to keep in the vicinity of the electrode the gas that has occurred, and enhance a generation efficiency of the high-order oxide.

Thereupon, as shown in FIG. 3, an electrolysis cell having a vapor phase electrolysis anode chamber installed therein has been devised. That is, a porous partitioning member 10 was installed into the anode chamber 1 of the two-chamber electrolysis cell of FIG. 1. That is, the anode chamber 1 was divided into a vapor phase electrolysis chamber 11 in which an anode electrode exists and a water passage chamber by means of the partitioning member 10. And, purer water supplied to the anode chamber 1 was prevented from directly entering the vapor phase electrolysis chamber 11. In FIG. 3, 1 is an anode chamber. 2 is an inlet of the anode chamber. 3 is an exit of the anode chamber. 4 is an anode electrode. 5 is a membrane. 6 is a cathode chamber. 7 is an inlet of the cathode chamber. 8 is an exit of the cathode chamber. 9 is a cathode electrode. As the partitioning member 10, for example, a porous film (or non-woven fabric) etc. having holes of which size is 0.5 to 5 mm opened therein can be employed. The electrolytic reaction product was prevented from directly being dissolved in anode water owing to existence of such a porous partitioning member 10. That is, the electrolytic reaction product stays in the vapor phase electrolysis anode chamber 11 for a time being. And, thereafter, it gradually diffuses into anode chamber supplying water. Additionally, employing a fluorine-based ion exchange membrane as the membrane 5 that contacts with the anode electrode 4 enhances an occurrence efficiency of ozone.

Further, a four-chamber electrolysis cell as shown in FIG. 4 was devised. This is configured so that the anode chamber of the three-chamber electrolysis cell of FIG. 2 is divided into two by means of a porous partitioning member 14. And, this prevents pure water supplied to the anode chamber from directly entering the vapor phase electrolysis chamber in the side in which the anode polarity exists. As a partitioning member, all the same, the material such as the porous film (or non-woven fabric) etc. having holes opened therein is employed. The electrolytic reaction product is prevented from directly being dissolved in anode water owing to existence of such a porous partitioning member. That is, the electrolytic reaction product stays in the vapor phase electrolysis anode chamber for a time being. And, thereafter, it gradually diffuses into anode chamber supplying water. In FIG. 4, 1 is a vapor phase electrolysis anode chamber. 2 is an inlet of an anode chamber. 3 is an exit of the anode chamber. 4 is an anode electrode. Each of 5 and 6 is a membrane. 7 is a cathode electrode. 8 is an exit of a cathode chamber. 9 is the cathode chamber. 10 is an inlet of the cathode chamber. 11 is an intermediate chamber. 12 is an inlet of the intermediate chamber. 13 is an exit of the intermediate chamber. 14 is the partitioning member.

Further, an electrolysis cell as well shown in FIG. 5 can be employed. In FIG. 5, 1 is an anode chamber. 2 is an inlet of the anode chamber. 3 is an exit of the anode chamber. 4 is an anode electrode support member. 5 is a membrane (fluorine-based cation exchange membrane). 6 is an anion exchange membrane. 7 is an inlet of an intermediate chamber. 8 is the intermediate chamber. 9 is an exit of the intermediate chamber. 10 is a cathode chamber. 11 is an inlet of the cathode chamber. 12 is an exit of the cathode chamber. 13 is a cathode electrode. 14 is a membrane (fluorine-based cation exchange membrane). 15 is an anode electrode (net-shape platinum electrode). A characteristic point of the electrolysis cell having this structure lies in the support member 4 of the anode electrode. The support member 4 has a structure shown in FIG. 6. Short pipes welded to the support member 4 support the anode electrode (net-shape platinum electrode) 15. Thus, the electrolytic product of the anode electrode 15 is not directly released into the anode chamber supplying water. That is, the electrolytic product is temporarily confined into a space between the support member 4 and the platinum electrode 15. As a result, the surface of the anode electrode (net-shape platinum electrode) 15 is covered with electrolysis generative gas. In this structure, employing the fluorine-based cation exchange membrane as the membrane 5 that contacts with the net-shape platinum electrode 15 enhances an occurrence efficiency of ozone. And, generating the high-order halogen acid necessitates halogen ion. Thus, a halogen salt is supplied to the intermediate chamber 8. Additionally, when the cation exchange membrane with a simplified shape is used, it is difficult to sufficiently supply halogen ion. Thus, the cation exchange membrane is preferably bored. As it is, when the cation exchange membrane is bored, a solution of the intermediate chamber migrates to the anode chamber. Thus, so as to prevent a solution of the intermediate chamber from migrating to the anode chamber while supplying a halogen ion, a negative-ion exchange membrane is preferably employed.

The present invention has been accomplished based upon the above-mentioned knowledge.

That is, the foregoing problem is solved by an aqueous solution containing at least one member selected from the group consisting of halogen acids and salts thereof, and further active oxygen, wherein the halogen acid is at least one member selected from the group consisting of hypochlorous acid, chlorous acid, chloric acid, and perchloric acid, wherein the total amount of the member selected from the group consisting of the halogen acid and salts thereof contained in the aqueous solution is 10 to 50,000 ppm, and wherein the total amount of the active oxygen contained in the aqueous solution is 0.1 to 1,000 ppm.

Further, the foregoing problem is solved by the above-mentioned aqueous solution, wherein the active oxygen is at least one member selected from the group consisting of hydrogen peroxide, hydroxyl radical, and superoxide anion.

Further, the foregoing problem is solved by the above-mentioned aqueous solution, wherein pH is 4 to 9.

Further, the foregoing problem is solved by the above-mentioned aqueous solution, wherein water obtained by carrying out electrolysis is employed.

Further, the foregoing problem is solved by the above-mentioned aqueous solution, wherein water obtained by subjecting a halogen-salt-containing aqueous solution to electrolysis is employed.

Further, the foregoing problem is solved by the above-mentioned aqueous solution, wherein water obtained by carrying out electrolysis in which a halogen salt has been supplied to a cathode chamber of an electrolysis cell (two-chamber electrolysis cell) including an anode chamber and a cathode chamber is employed.

Further, the foregoing problem is solved by the above-mentioned aqueous solution, wherein water obtained by carrying out electrolysis in which a halogen salt has been supplied to an intermediate chamber of an electrolysis cell (three-chamber electrolysis cell) including an anode chamber, an intermediate chamber, and a cathode chamber is employed.

Further, the foregoing problem is solved by the above-mentioned aqueous solution, wherein water obtained by carrying out a vapor phase electrolysis oxidation in which, in an electrolysis cell (three-chamber electrolysis cell) including a cathode chamber and an anode chamber having a porous member laid out inside it, a halogen salt has been supplied to the foregoing cathode chamber is employed.

Further, the foregoing problem is solved by the above-mentioned aqueous solution, wherein water obtained by carrying out a vapor phase electrolysis oxidation in which, in an electrolysis cell (four-chamber electrolysis cell) including a cathode chamber, an intermediate chamber, and an anode chamber having a porous member laid out inside it, a halogen salt has been supplied to the foregoing intermediate chamber is employed.

Further, the foregoing problem is solved by the above-mentioned aqueous solution that is employed for disinfection.

Further, the foregoing problem is solved by a disinfecting method of carrying out disinfection by employing the above-mentioned aqueous solution.

Further, the foregoing problem is solved by the above-mentioned aqueous solution that is employed for cleaning.

Further, the foregoing problem is solved by a cleaning method of carrying out cleaning by employing the above-mentioned aqueous solution.

Further, the foregoing problem is solved by a method of prolonging life duration of residual chlorine in an aqueous solution, said method comprising:

causing the member selected from the group consisting of at least one halogen acid selected from the group consisting of hypochlorous acid, chlorous acid, chloric acid, and perchloric acid, and salts thereof to be contained in water so that the total amount thereof is a ratio of 10 to 50,000 ppm, and

causing active oxygen to be contained in water so that the total amount thereof is a ratio of 0.1 to 1,000 ppm.

Further, the foregoing problem is solved by a method of prolonging life duration of residual chlorine in the above-mentioned aqueous solutions, said method comprising:

causing the member selected from the group consisting of at least one halogen acid selected from the group consisting of hypochlorous acid, chlorous acid, chloric acid, and perchloric acid, and salts thereof to be contained in water so that the total amount thereof is a ratio of 10 to 50,000 ppm, and

causing active oxygen to be contained in water so that the total amount thereof is a ratio of 0.1 to 1,000 ppm.

### AN ADVANTAGEOUS EFFECT OF THE INVENTION

It is well-known that the hypochlorite (ClO⁻) exhibits an effect of disinfection (sterilization).

However, when life duration of ClO⁻ is short, an effect of disinfection/sterilization declines with a lapse of time. Thus, it is important to prolong life duration of ClO⁻.

The present invention makes it possible to maintain a high ClO⁻ concentration that exhibits the above-mentioned disinfection/sterilization effect over a long period. Thus, with the present invention, an effect as a disinfectant solution (sterilization solution) is sufficiently exhibited. Further, a cleaning effect as well is sufficiently exhibited.

### BRIEF DESCRIPTION OF THE DRAWINGS

FIG. 1 is a schematic view of a two-chamber electrolysis cell.

FIG. 2 is a schematic view of a three-chamber electrolysis cell.

FIG. 3 is a schematic view of a three-chamber vapor-phase electrolysis cell.

FIG. 4 is a schematic view of a fourth-chamber vapor-phase electrolysis cell.

FIG. 5 is a schematic view of a fourth-chamber vapor-phase electrolysis cell.

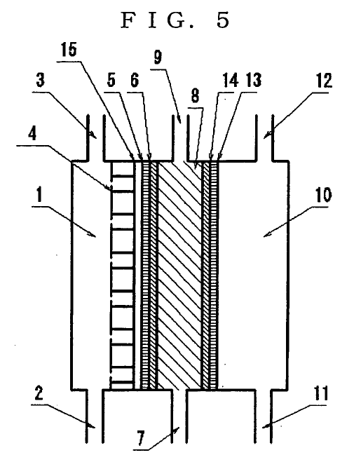

FIG. 6 is a schematic view of a support member.

FIG. 7 is a graph of a residual chlorine concentration.

FIG. 8 is a graph of a residual chlorine concentration.

FIG. 9 is a graph of a residual chlorine concentration.

### BEST MODE FOR CARRYING OUT THE INVENTION

The aqueous solution in accordance with the present invention contains at least one member selected from the group consisting of the halogen acids and salts thereof, and further active oxygen. The halogen acid is at least one member selected from the group consisting of hypochlorous acid, chlorous acid, chloric acid, and perchloric acid. The total amount of the member selected from the group consisting of the halogen acid and salts thereof that is contained in the aqueous solution is 10 to 50,000 ppm (in particular, more preferably, 10 to 300 ppm). That is, specifying the total amount of the member selected from the group consisting of the halogen acid and salts thereof as mentioned above allows the residual chlorine having a high concentration to be maintained. Further, the total amount of the active oxygen that is contained in the aqueous solution is 0.1 to 1,000 ppm (in particular, preferably, 1 to 100 ppm). That is, specifying the total amount of the active oxygen as mentioned above allows the residual chlorine having a high concentration to be maintained. The foregoing active oxygen is any member selected from the group consisting of, for example, hydrogen peroxide, hydroxyl radical, and superoxide anion. pH of the aqueous solution is preferably 4 to 9 (in particular, more preferably, 6 to 8). The water being employed for the aqueous solution is, for example, water obtained by carrying out electrolysis. In particular, it is water obtained by subjecting the halogen-salt-containing aqueous solution to electrolysis. Above all, it is water obtained by carrying out electrolysis in which a halogen salt has been supplied to the cathode chamber of the two-chamber electrolysis cell (the electrolysis cell including the anode chamber and the cathode chamber). Or, it is water obtained by carrying out electrolysis in which a halogen salt has been supplied to the intermediate chamber of the three-chamber electrolysis cell (the electrolysis cell including the anode chamber, the intermediate chamber, and the cathode chamber). Or, it is water obtained by carrying out a vapor phase electrolysis oxidation in which a halogen salt has been supplied to the cathode chamber of the three-chamber electrolysis cell (the electrolysis cell including the cathode chamber and the anode chamber having the porous member laid out inside it). Or, it is water obtained by carrying out a vapor phase electrolysis oxidation in which a halogen salt has been supplied to the intermediate chamber of the four-chamber electrolysis cell (the electrolysis cell including the cathode chamber, the intermediate chamber, and the anode chamber having the porous member laid out inside it).

The above-mentioned aqueous solution is employed, in particular, for disinfection and/or cleaning.

The present invention is a disinfecting method of carrying out disinfection by employing the above-mentioned aqueous solution.

The present invention is a cleaning method of carrying out cleaning by employing the above-mentioned aqueous solution.

The present invention is a method of prolonging life duration of residual chlorine in an aqueous solution. In particular, it is a method of prolonging life duration of the residual chlorine in the above-mentioned aqueous solutions. And, it includes a step of causing the member selected from the group consisting of at least one halogen acid selected from the group consisting of hypochlorous acid, chlorous acid, chloric acid, and perchloric acid, and salts thereof to be contained in water so that the total amount thereof is a ratio of 10 to 50,000 ppm (preferably, 10 to 300 ppm). Yet, it includes a step of causing active oxygen to be contained in water so that the total amount thereof is a ratio of 0.1 to 1,000 ppm (preferably, 1 to 100 ppm).

Hereinafter, the present invention will be specifically explained.

### Example 1

KClO₃ was caused to be dissolved in pure water. Citric acid was added to this KClO₃ aqueous solution. With this, pH was adjusted to approx. 4. The residual chlorine concentration of this KClO₃ aqueous solution with pH 4 was measured. Further, a H₂O₂ aqueous solution was added to the above-mentioned KClO₃ aqueous solution with pH 4, and the residual chlorine concentration was measured.

This measurement (measurement with the KI method) is shown in Table-1 mentioned below.

TABLE 1

| | Elapsing | | | | | | |
|---|---|---|---|---|---|---|---|
| | time | Concentration (ppm) | | | | | |
| Potassium | | 125 | 125 | 125 | 125 | 125 | 125 |
| chlorate | | | | | | | |
| Hydrogen | | 0 | 30 | 60 | 120 | 240 | 480 |
| peroxide | | | | | | | |
| Residual | 0 Day | 0 | 5 | 25 | 30 | 30 | 125 |
| chlorine | 7 Days | 0 | 200 | 250 | 300 | 300 | 350 |
| | 14 days | 0 | 200 | 250 | 300 | 300 | 350 |
| | 30 days | 0 | 200 | 250 | 300 | 300 | 350 |

It can be seen from this table-1 that causing the KClO₃ aqueous solution to contain H₂O₂ enables a high residual chlorine concentration therein to be kept even though days have elapsed. That is, the disinfection/sterilization effect is kept over a long period.

Additionally, the similar effect is exhibited with the case as well of employing a hydroxyl-radical-containing aqueous solution, and a superoxide-anion-containing aqueous solution instead of a hydrogen-peroxide-containing aqueous solution. This is made understandable by the cases of example 2 and examples after it (the case of employing the electrolytic water).

### Example 2

KClO₃ was caused to be dissolved in pure water. Citric acid was added to this KClO₃ aqueous solution. With this, pH was adjusted to approx. 4. Besides it, NaClO was added to this KClO₃ aqueous solution. And, the residual chlorine concentration was measured. As a result, no significant difference with the H₂O₂ aqueous solution existed.

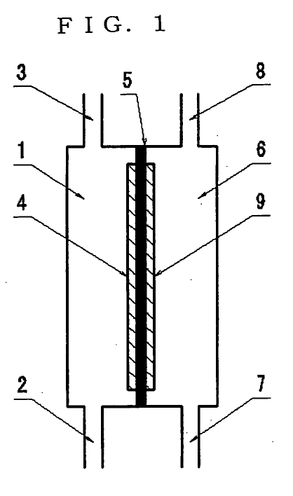

Next, the anodic electrolytic water generated by employing the two-chamber electrolysis cell shown in FIG. 1 was employed instead of the above-mentioned pure water. The two-chamber electrolysis cell is configured so as to employ a 80-mesh net of a platinum electrode (size of the electrode 80 mm×60 mm) as an anode electrode, and a titanium electrode (size of the electrode 80 mm×60 mm) as a cathode electrode, and to employ a fluorine-based cation exchange membrane as a membrane for separating the anode chamber from the cathode chamber. And, pure water was supplied to the cathode chamber and the anode chamber.

NaClO was caused to be dissolved in this anodic electrolytic water in an amount of 80 ppm. Besides it, KClO₃ was added in an amount of 125 ppm. Further, citric acid was added to adjust pH to approx. 6.

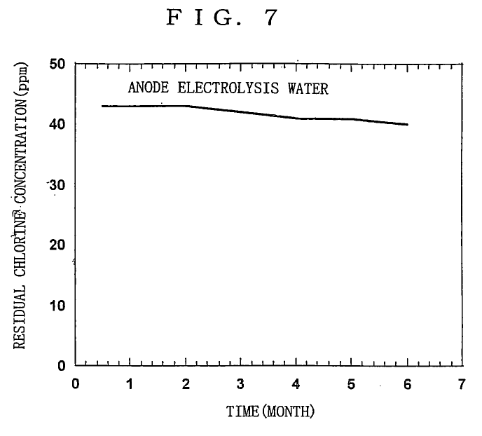

The residual chlorine concentration of this aqueous solution containing NaClO and KClO₃ (water; anodic electrolytic water) was measured (measurement with the KI method), so its result is shown in FIG. 7.

It can be seen from this FIG. 7 that the residual chlorine concentration of the aqueous solution containing NaClO and KClO₃ has been kept at a high concentration level over a long period.

### Example 3

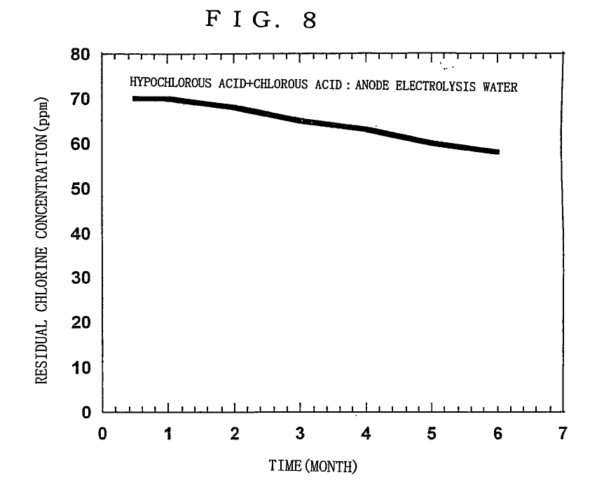

NaClO and KClO₂ were caused to be dissolved in the anodic electrolytic water of the example 2 in an amount of 40 ppm and 100 ppm, respectively. Besides it, citric acid was added to adjust pH to approx. 6. The residual chlorine concentration of this aqueous solution containing NaClO and KClO₂ was measured (measurement with the KI method), so its result is shown in FIG. 8.

It can be seen from this FIG. 8 that the residual chlorine concentration of the aqueous solution containing NaClO and KClO₂ has been kept at a high concentration level over a long period.

### Example 4

In this example, pure water and the anodic electrolytic water explained in the example 2 were employed as water. And, the aqueous solution caused to contain H₂O₂ (150 ppm) and HClO₄ (125 ppm) were prepared.

The residual chlorine concentration of this aqueous solution was measured (measurement with the KI method), so its result is shown in Table-2.

TABLE 2

| | | | Anodic |
|---|---|---|---|
| | | | electrolytic |
| | Elapsing time | Pure water | water |
| Residual | O month | 30 | 60 |
| chlorine | 1 month | 25 | 60 |
| concentration | 2 months | 20 | 60 |
| (ppm) | 3 months | 15 | 55 |
| | 4 months | 5 | 55 |

It can be also seen from this table-2 that the residual chlorine concentration in the case of employing of the anodic electrolytic water is higher.

### Example 5

In this example, pure water and the anodic electrolytic water explained in the example 2 were employed as water. And, the aqueous solution caused to contain KClO₂ (150 ppm) and HClO₄ (62.5 ppm) were prepared.

The residual chlorine concentration of this aqueous solution was measured (measurement with the KI method), so its result is shown in Table-3.

TABLE 3

| | | | Anodic |
|---|---|---|---|
| | | | electrolytic |
| | Elapsing time | Pure water | water |
| Residual | O month | 30 | 60 |
| chlorine | 1 month | 25 | 60 |
| concentration | 2 months | 20 | 60 |
| (ppm) | 3 months | 15 | 55 |
| | 4 months | 5 | 55 |

It can be also seen from this table-3 that the residual chlorine concentration in the case of employing of the anodic electrolytic water is higher.

### Example 6

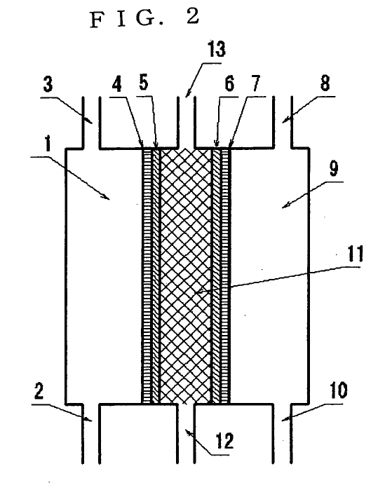

The electrolytic cathode water generated by employing the three-chamber electrolysis cell shown in FIG. 2 was employed. The three-chamber electrolysis cell is configured so as to employ a 80-mesh net of a platinum electrode (size of the electrode 80 mm×60 mm) as an anode electrode, and a titanium electrode (size of the electrode 80 mm×60 mm) as a cathode electrode, and to employ a fluorine-based cation exchange membrane as a membrane for separating the anode chamber from the cathode chamber. And, saturated saline water was supplied to the intermediate chamber, and pure water was supplied to the cathode chamber and the anode chamber. KClO₃ was caused to be dissolved in this electrolytic cathode water in an amount of 125 ppm. Besides it, citric acid was added to adjust pH to approx. 4.

The residual chlorine concentration of this KClO₃-containing aqueous solution (water; electrolytic cathode water) was measured (measurement with the KI method). As a result, it was conformed that the residual chlorine concentration was kept at a high concentration level over a long period.

### Example 7

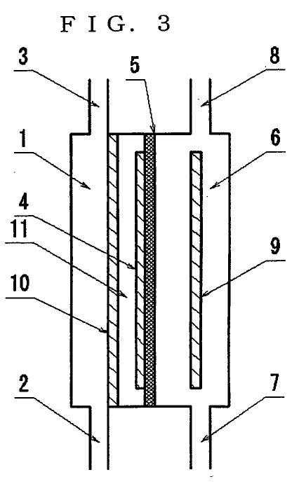

In this example, the anodic electrolytic water obtained by the electrolysis device of FIG. 3 was employed instead of the anodic electrolytic water of the example 2. And, the residual chlorine concentration was measured.

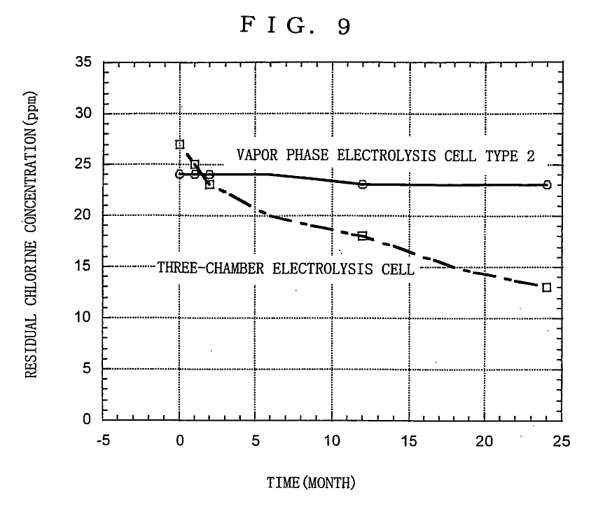

Its result is shown in FIG. 9. It can be seen from FIG. 9 that the anodic electrolytic water by the electrolysis device including the electrolysis cell having the vapor phase electrolysis anode chamber installed therein is preferably employed notwithstanding the identical anodic electrolytic water.

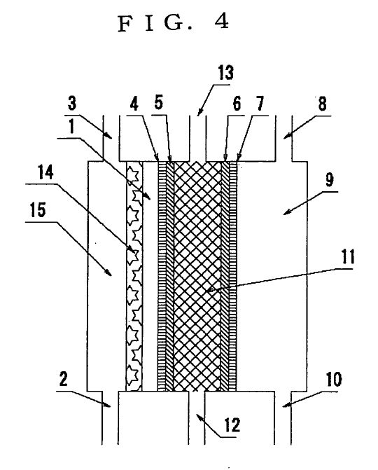

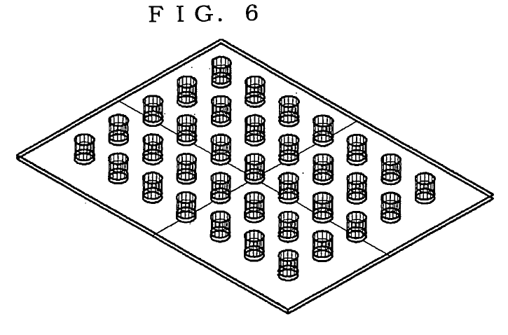

Further, the anodic electrolytic water obtained by employing the electrolysis devices of a FIG. 4 type and a FIG. 6 type was employed to prepare the aqueous solution likewise, and the residual chlorine concentration was measured. As a result, all the same, it can be seen that the anodic electrolytic water by the electrolysis device including the electrolysis cell having the vapor phase electrolysis anode chamber installed therein is preferably employed.

### HOW THE INVENTION IS CAPABLE OF INDUSTRIAL EXPLOITATION

The present invention is effectively employed for a disinfection field and a cleaning field.

1.-14. (canceled)

15. An aqueous solution containing water, at least one member selected from the group consisting of halogen acids and salts thereof, and further active oxygen, said aqueous solution being employed for disinfection and/or cleaning:
wherein said water is vapor-phase electrolytic anodic water obtained by an electrolysis device comprising an electrolysis cell having a vapor phase electrolysis anode chamber;
wherein said halogen acid is at least one member selected from the group consisting of hypochlorous acid, chlorous acid, chloric acid, and perchloric acid;
wherein the total amount of the member selected from the group consisting of said halogen acid and salts thereof contained in said aqueous solution is 10 to 50,000 ppm,
wherein the total amount of the active oxygen that is contained in said aqueous solution is 0.1 to 1,000 ppm.

16. The aqueous solution as claimed in claim 15, wherein said water is vapor-phase electrolytic anodic water obtained by carrying out a vapor phase electrolysis oxidation under a condition that, in an electrolysis cell comprising an anode chamber partitioned inside by a porous member, and a cathode chamber, a halogen salt is supplied to said cathode chamber.

17. The aqueous solution as claimed in claim 15, wherein said water is vapor-phase electrolytic anodic water obtained by carrying out a vapor phase electrolysis oxidation under a condition that, in an electrolysis cell comprising an anode chamber partitioned inside by a porous member, an intermediate chamber, and a cathode chamber, a halogen salt is supplied to said cathode chamber.

18. The aqueous solution as claimed in claim 15, wherein said active oxygen is at least one member selected from the group consisting of hydrogen peroxide, hydroxyl radical, and superoxide anion.

19. The aqueous solution as claimed in claim 15, wherein pH of said aqueous solution is 4 to 9.

20. A method of producing the aqueous solution as claimed in claim 15, said aqueous solution being employed for disinfection and/or cleaning, said method comprising:
obtaining vapor-phase electrolytic anodic water by employing an electrolysis device comprising an electrolysis cell comprising a vapor phase electrolysis anode chamber;
causing the member selected from the group consisting of at least one halogen acid selected from the group consisting of hypochlorous acid, chlorous acid, chloric acid, and perchloric acid, and salts thereof to be contained in said vapor-phase electrolytic anodic water so that the total amount thereof is a ratio of 10 to 50,000 ppm; and
causing the active oxygen to be contained in said vapor-phase electrolytic anodic water so that the total amount thereof is a ratio of 0.1 to 1,000 ppm.

21. A disinfecting method of carrying out disinfection by employing the aqueous solution as claimed in claim 15.

22. A cleaning method of carrying out cleaning by employing the aqueous solution as claimed in claim 15.

23. A method of prolonging life duration of residual chlorine in an aqueous solution, said method comprising:
causing a member selected from the group consisting of at least one halogen acid selected from the group consisting of hypochlorous acid, chlorous acid, chloric acid, and perchloric acid, and salts thereof to be contained in vapor-phase electrolytic anodic water obtained by an electrolysis device comprising an electrolysis cell having a vapor phase electrolysis anode chamber so that the total amount thereof is a ratio of 10 to 50,000 ppm; and
causing active oxygen to be contained so that the total amount thereof is a ratio of 0.1 to 1,000 ppm.

24. The method of prolonging life duration of residual chlorine in the aqueous solution as claimed in claim 23, wherein said vapor-phase electrolytic anodic water is vapor-phase electrolytic anodic water obtained by carrying a vapor phase electrolysis oxidation under a condition that, in an electrolysis cell comprising an anode chamber partitioned inside by a porous member, and a cathode chamber, a halogen salt is supplied to said cathode chamber.

25. The method of prolonging life duration of residual chlorine in the aqueous solution as claimed in claim 23, wherein said vapor-phase electrolytic anodic water is vapor-phase electrolytic anodic water obtained by carrying out a vapor phase electrolysis oxidation under a condition that, in an electrolysis cell comprising an anode chamber partitioned inside by a porous member, an intermediate chamber, and a cathode chamber, a halogen salt is supplied to said cathode chamber.

26. The method of prolonging life duration of residual chlorine in the aqueous solution as claimed in claim 23, wherein said active oxygen is at least one member selected from the group consisting of hydrogen peroxide, hydroxyl radical, and superoxide anion.

27. A method of producing an aqueous solution, said aqueous solution being employed for disinfection and/or cleaning, said method comprising:
obtaining vapor-phase electrolytic anodic water by employing an electrolysis device comprising an electrolysis cell comprising a vapor phase electrolysis anode chamber;
causing the member selected from the group consisting of at least one halogen acid selected from the group consisting of hypochlorous acid, chlorous acid, chloric acid, and perchloric acid, and salts thereof to be contained in said vapor-phase electrolytic anodic water so that the total amount thereof is a ratio of 10 to 50,000 ppm; and
causing the active oxygen to be contained in said vapor-phase electrolytic anodic water so that the total amount thereof is a ratio of 0.1 to 1,000 ppm.

28. A method of producing the aqueous solution as claimed in claim 27, wherein said water is vapor-phase electrolytic anodic water obtained by carrying out a vapor phase electrolysis oxidation under a condition that, in an electrolysis cell comprising an anode chamber partitioned inside by a porous member, and a cathode chamber, a halogen salt is supplied to said cathode chamber.

29. A method of producing the aqueous solution as claimed in claim 27, wherein said water is vapor-phase electrolytic anodic water obtained by carrying out a vapor phase electrolysis oxidation under a condition that, in an electrolysis cell comprising an anode chamber partitioned inside by a porous member, an intermediate chamber, and a cathode chamber, a halogen salt is supplied to said cathode chamber.

30. A method of producing the aqueous solution as claimed in claim 27, wherein said active oxygen is at least one member selected from the group consisting of hydrogen peroxide, hydroxyl radical, and superoxide anion.

31. A method of producing the aqueous solution as claimed in claim 27, wherein pH of said aqueous solution is 4 to 9.
